(19) United States
(12) Patent Application Publication
(10) Pub. No.: US 2014/0069421 A1
(43) Pub. Date: Mar. 13, 2014

(54) MEDICAL ORAL AIRWAY INSTRUMENT

(71) Applicant: PO-YU KUO, New Taipei City (TW)

(72) Inventor: PO-YU KUO, New Taipei City (TW)

(21) Appl. No.: 13/612,635

(22) Filed: Sep. 12, 2012

(51) Int. Cl. A61M 16/04

(52) U.S. Cl. 128/200.26

(57) ABSTRACT

The present invention discloses a medical oral airway instrument. A bite portion of the oral airway instrument is provided with a soft pad piece made by soft plastic, soft rubber or soft silicon of medical use. The soft pad piece can be a soft washer or two cushions, allowing a patient to feel more uncomfortable and protecting the patient's teeth from injury, when the patient bites at the soft pad piece of the oral airway instrument.

### BACKGROUND OF THE INVENTION

a) Field of the Invention

The present invention relates to a medical oral airway instrument, and more particularly to a medical oral airway instrument, wherein a soft pad piece is directly covered on a bite portion of the oral airway instrument to reduce uncomfortableness as the oral airway instrument is bitten by a patient's teeth.

b) Description of the Prior Art

With reference to FIG. 6, a conventional oral cavity insertion instrument is formed by direct injection molding to hard plastic. An insertion end 101 at one end of an oral airway instrument 100 is inserted into the oral cavity of a patient, and the patient's mouth directly bites at a bite portion 102 in the middle of the oral airway instrument 100, whereas a baffle 103 at the other end stops at a front end of the patient's mouth, with that a vent hole 104 in the center of the baffle 103 provides for insertion of a hose (not shown in the drawings). As the bite portion 102 of the oral airway instrument 100 is harder, the patient will usually feel uncomfortable with his or her teeth and the teeth may be even injured, when the patient bites at the bite portion 102. Moreover, as the insertion end 101 of the oral airway instrument 100 is also made by a harder material, the patient will feel uncomfortable with his or her throat when the oral airway instrument 100 is inserted into his or her oral cavity.

### SUMMARY OF THE INVENTION

Accordingly, the primary object of the present invention is to provide a medical oral airway instrument, wherein a bite portion of the oral airway instrument is covered with a soft pad piece made by soft plastic, soft rubber or soft silicon of medical use, and the soft pad piece can be a soft washer or cushions, so that when the oral airway instrument is inserted into a patient's oral cavity, the soft pad piece can allow the patient to feel more comfortable and can even protect the patient's teeth from injury, when the patient bites at the bite portion.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view of a first embodiment of the present invention;

FIG. 2 shows an exploded view of the first embodiment of the present invention;

FIG. 3 shows a cross-sectional view of the first embodiment of the present invention;

FIG. 4 shows an exploded view of a second embodiment of the present invention;

FIG. 5 shows a schematic view of a third embodiment of the present invention;

FIG. 5-1 shows a schematic view of a fourth embodiment of the present invention; and

FIG. 6 shows a schematic view of a prior art.

### DETAILED DESCRIPTION OF THE PREFERRED EMBODIMENTS

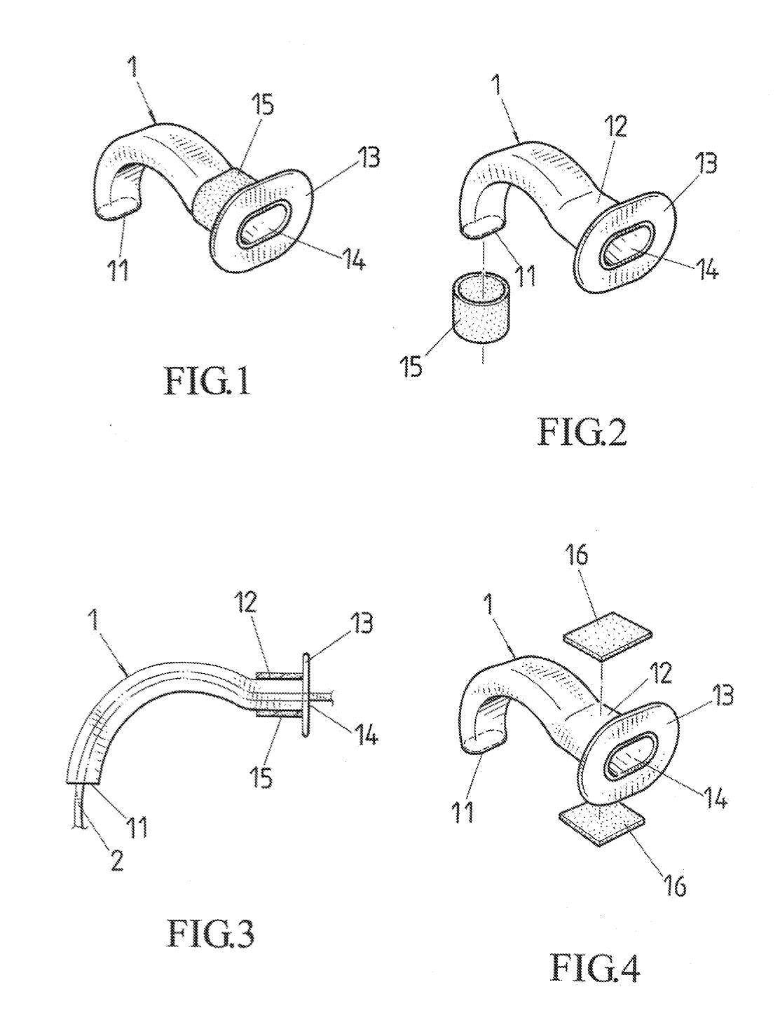

With reference to FIGS. 1 to 3, as the prior art, an insertion end 11 of an oral airway instrument 1 of the present invention is inserted into a patient's oral cavity. A bite portion 12 is at the middle of the oral airway instrument 1 and a baffle 13 at the other end stops at a front end of the patient's mouth. A vent hole 14 in the center of the baffle 14 allows an ordinary hose (as shown in FIG. 3) to be deeply inserted into the patient's throat. The present invention is characterized as below.

The bite portion 12 of the oral airway instrument 1 is sheathed with a soft pad piece 15 made by soft plastic, soft rubber or soft silicon of medical use, with that an embodiment in the drawings is a soft washer. With reference to FIG. 4, it shows an exploded view of a second embodiment of the present invention, wherein the soft pad piece 16 is two soft plate-typed cushions put above and below the bite portion 12. The soft pad piece 16 can be attached on the bite portion 12 by medical glue, or the soft pad piece 16 and the bite portion 12, which are of different hardness, can be extruded and formed integrally by a plastic injection molding machine.

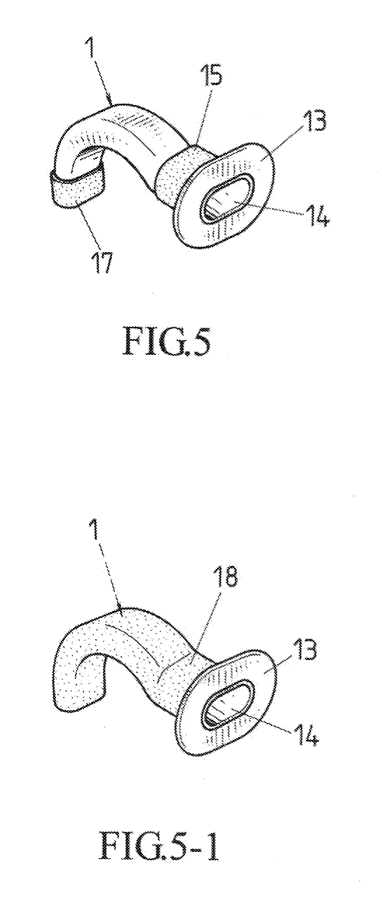

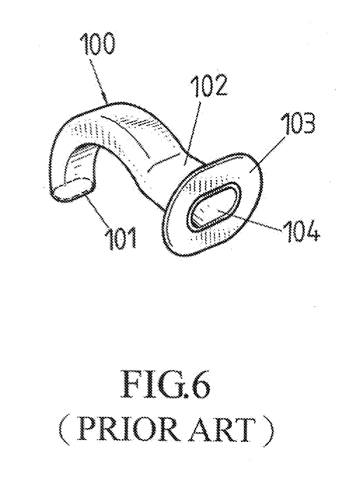

With reference to FIG. 5, it shows a schematic view of a third embodiment of the present invention, wherein the insertion end 11 of the oral airway instrument 1 of the present invention can be also installed with a soft pad piece 17 to reduce uncomfortableness of the patient, when the oral airway instrument 1 is inserted into the patient's oral cavity. With reference to FIG. 5-1, it shows a schematic view of a fourth embodiment of the present invention, in order to allow the patient to be safer and to protect the patient's teeth and oral cavity from injury, the entire oral airway instrument 1 is sheathed with a soft pad piece 18 made by soft plastic, soft rubber or soft silicon of medical use.

In conclusion, the present invention provides a medical oral airway instrument, wherein the bite portion of the oral airway instrument is installed with a soft pad piece, allowing the patient to feel more uncomfortable and protecting the patient's teeth from injury, when the patient bites at the bite portion.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claim is:

1. A medical oral airway instrument, wherein an end of the medical airway instrument is an insertion end, a bite portion is at the middle of the medical air way instrument and a baffle is at the other end, with that a vent hole is in the center of the baffle for insertion of a hose and the bite portion of the oral airway instrument is provided with a soft pad piece.

2. The medical oral airway instrument according to claim 1, wherein the soft pad piece is a soft washer.

3. The medical oral airway instrument according to claim 1, wherein the soft pad piece is two cushions.

4. The medical oral airway instrument according to claim 1, wherein the insertion end of the oral airway instrument is provided with a soft pad piece.

5. The medical oral airway instrument according to claim 1, wherein the entire oral airway instrument is sheathed with a soft pad piece.
